(19) United States
(12) Patent Application Publication
(10) Pub. No.: US 2024/0358432 A1
(43) Pub. Date: Oct. 31, 2024

(54) MEDICAL DEVICES AND RELATED METHODS FOR DELIVERING ENERGY AND/OR FLUID

(71) Applicant: Boston Scientific Scimed, Inc., Maple Grove, MN (US)

(72) Inventors: Austin Grant JOHNSON, Worcester, MA (US); Kurt Nicholas ROBAKIEWICZ, Upton, MA (US); Allison KUMARASENA, Wellesley, MA (US); Ryan V. WALES, Northborough, MA (US)

(73) Assignee: Boston Scientific Scimed, Inc., Maple Grove, MN (US)

(21) Appl. No.: 18/642,924

(22) Filed: Apr. 23, 2024

Related U.S. Application Data
(60) Provisional application No. 63/498,381, filed on Apr. 26, 2023.

(51) Int. Cl. A61B 18/14; A61B 17/00; A61B 18/00; A61M 25/01; A61M 25/10

(52) U.S. Cl. CPC A61B 18/1492; A61B 2017/00327; A61B 2018/00285; A61B 2018/00601; A61B 2018/0091; A61M 25/0136; A61M 25/0147; A61M 25/1018

(57) ABSTRACT

A medical device includes a handle, a shaft, an electrode, and at least one inflatable balloon. The handle includes an electrical connection and a balloon fluid port. The shaft extends from a distal portion of the handle. The electrode is positioned at a distal end of the shaft. The electrode is electrically connected to the electrical connection via one or more conductive elements extending through the handle and the shaft. The at least one inflatable balloon is positioned on a portion of shaft. The at least one inflatable balloon is fluidly coupled to the balloon fluid port via one or more tubes extending through the handle and the shaft.

### CROSS-REFERENCE TO RELATED APPLICATIONS

This application claims priority to U.S. Provisional Application No. 63/498,381, filed on Apr. 26, 2023, which is incorporated by reference herein in its entirety.

### TECHNICAL FIELD

Various aspects of this disclosure generally relate to medical devices and related methods for delivering energy and/or fluid. Embodiments of the disclosure relate to medical devices and related methods of treating tissue by delivering electrical energy to or into tissue and/or injecting fluid into, under, and/or around tissue with a treatment device. Moreover, embodiments of the disclosure relate to medical devices and related methods of treating tissue that includes deflecting a distal portion of the medical device and/or inflating a balloon at the distal portion of the medical device.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Many of these procedures involve delivering energy to tissue of an organ or a gland to treat lesions (e.g., tumors), infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy, Peroral Endoscopic Myotomy (POEM), etc. In particular, such procedures may be carried out by inserting an insertion device into a subject's body through a surgical incision, or via a natural anatomical orifice (e.g., mouth, vagina, or rectum), and performing the procedure or operation at a treatment site with an auxiliary device inserted through the insertion device. Alternatively, the auxiliary device may be delivered to the treatment site without the insertion device.

In some procedures, a distal portion of a medical device is positioned between layers of tissue, for example, to perform a “third space” procedure. In some aspects, a distal portion of the medical device is positioned between the mucosa (or mucosal) and the muscularis layers in a portion of the gastrointestinal (GI) tract to reach a target or treatment site. Oftentimes, the positioning is achieved by (1) injecting a lifting agent into the submucosa to separate the mucosa layer from the muscularis layer and (2) utilizing a cutting knife to cut through the submucosa. These procedures may require the use of multiple medical devices (e.g., an injection needle and a cutting knife). Using multiple medical devices may require a larger insertion device and/or swapping out the medical devices, which may increase the procedure duration, require multiple users, and/or otherwise negatively affect the procedure. Moreover, medical devices often used in the “third space” procedures often only include one degree of navigational freedom. In some aspects, the separation of the layers of tissue may dissipate over time or during the procedure, increasing a risk that the medical device(s) may inadvertently contact tissue or other aspects of the treatment site. These concerns may increase the duration, costs, and risks of the medical procedure.

The devices and methods of this disclosure may rectify some of the deficiencies described above or address other aspects of the art.

### SUMMARY

Examples of this disclosure relate to, among other things, medical devices and methods for performing one or more medical procedures. For example, this disclosure relates to medical devices and methods for performing one of more procedures between layers of tissue. Additionally, in some examples, this disclosure relates to medical devices and methods for delivering energy (e.g., for cutting, cauterizing, perforating, puncturing, or otherwise treating tissue) and/or delivering fluid to a treatment site. Furthermore, in some examples, this disclosure relates to medical devices and methods for inflating and/or deflating one or more balloons on a distal end of the medical device. Moreover, in some examples, this disclosure relates to medical devices and methods for articulating, deflecting, or otherwise maneuvering a distal portion of the medical device. Each of the examples disclosed herein may include one or more of the features described in connection with any of the other disclosed examples.

In one example, a medical device may include a handle, a shaft, an electrode, and at least one inflatable balloon. The handle may include an electrical connection and a balloon fluid port. The shaft may extend from a distal portion of the handle. The electrode may be positioned at a distal end of the shaft. The electrode may be electrically connected to the electrical connection via one or more conductive elements extending through the handle and the shaft. The at least one inflatable balloon may be positioned on a portion of shaft. The at least one inflatable balloon may be fluidly coupled to the balloon fluid port via one or more tubes extending through the handle and the shaft.

The medical device may include one or more of the following features. The handle may further include one or more deflection elements, and at least a portion of the shaft may be deflectable via movement of the one or more deflection elements. The one or more deflection elements may be each coupled to a wheel that is connected to one or more pull wires that extend through the handle and are secured to one or more interior portions of the shaft such that movement of the one or more deflection elements may urge the one or more pull wires proximally to deflect a portion of the shaft. The one or more deflection elements may include two coaxial deflection knobs. Each of the two deflection knobs may control a deflection of the portion of the shaft in substantially perpendicular planes. The portion of the shaft may be deflectable in a substantially semi-spherical range of motion. The portion of the shaft may be deflectable with the at least one inflatable balloon inflated. The handle may further include one or more locks to secure a position of one or more of the two deflection knobs.

The electrode may include an electrode lumen. The handle may further include an electrode fluid port. The electrode fluid port may be fluidly coupled to the electrode lumen via one or more electrode fluid tubes that are configured to deliver a fluid to the electrode lumen from the electrode fluid port. The electrode may include an electrode shaft and a distal end. The distal end may be wider relative to a longitudinal axis than the electrode shaft. The handle may further include a trigger. The electrode may be extendable distally and retractable proximally relative to the distal end of the shaft via actuation of the trigger. The distal end of the electrode may remain positioned distally of the distal end of the shaft when the electrode is in a retracted position. The trigger may be positioned on a distal portion of the handle. The electrode fluid port may be positioned on a distal portion of the handle. The electrical connection and the balloon fluid port may be positioned on a proximal portion of the handle.

An entirety of the electrode may be conductive. The at least one inflatable balloon may be fluidly coupled to one or more tubes via at least one balloon hole in an outer surface of a distal portion of the shaft. The at least one balloon hole may include two balloon holes positioned on circumferentially opposite sides of the shaft. The medical device may be configured to perform a third space endoscopy procedure.

In another aspect, a medical device may include a handle, a shaft extending from a distal portion of the handle, and an electrode positioned at a distal end of the shaft. The handle may include an electrical connection and one or more deflection controls. The electrode may be electrically connected to the electrical connection via one or more conductive elements extending through the handle and the shaft. At least a portion of the shaft may be deflectable via movement of the one or more deflection controls.

The medical device may include one or more of the following features. The electrode may include an electrode shaft and a distal end. The distal end may be wider relative to a longitudinal axis than the electrode shaft. The electrode may include an electrode lumen. The handle may further include an electrode fluid port. The electrode fluid port may be fluidly coupled to the electrode lumen via one or more electrode fluid tubes that are configured to deliver a fluid to the electrode lumen from the electrode fluid port. The medical device may further include at least one inflatable balloon positioned on a distal portion of the shaft. The handle may further include a balloon fluid port. The at least one inflatable balloon may be fluidly coupled to the balloon fluid port via one or more tubes extending through the handle and the shaft.

In yet another aspect, a method of treating a treatment site may include delivering a distal end of a shaft of a medical device to a treatment site. The shaft may include a deflectable portion, and an electrode may be movably positioned at the distal end of the shaft. The method may also include delivering energy to the treatment site to cut, puncture, or perforate one or more layers of tissue at the treatment site. The method may further include positioning the distal end of the shaft between layers of tissue, and expanding one or more balloons to separate layers of tissue. The one or more balloons may be positioned on a distal portion of the shaft and may be fluidly connected to one or more balloon fluid ports via one or more balloon fluid tubes and one or more balloon holes. The method may also include positioning and energizing the electrode to deliver energy to one or more portions of the treatment site. The one or more portions of the treatment site may be between separated layers of tissue.

The method may further include one or more of the following features. The electrode may include an electrode lumen fluidly connected to a fluid source. The method may further include, before positioning the distal end of the shaft between layers of tissue, injecting or otherwise delivering fluid to the treatment site through the electrode lumen to separate layers of tissue. The method may further include, before positioning and energizing the electrode to delivery energy to one or more portions of the treatment site, articulating or deflecting the distal end of the shaft to further separate the layers of tissue.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 illustrates a perspective view of an exemplary medical device, including an enlarged view of a distal portion of the medical device in a first configuration, according to aspects of this disclosure.

FIG. 2 illustrates the distal portion of the medical device of FIG. 1 in a second configuration, according to aspects of this disclosure.

FIG. 3 illustrates a schematic view of the medical device of FIG. 1 in the second configuration, according to aspects of this disclosure.

FIG. 4 is a flow diagram of an exemplary method, according to aspects of this disclosure.

### DETAILED DESCRIPTION

Examples of the disclosure include devices and methods for one or more of: facilitating and improving the efficacy, efficiency, and safety of treating and/or manipulating tissue when, for example, applying electrical energy to tissue with an electrode; delivering fluid into, under, and/or around tissue during a medical procedure through the distal end of the electrode; and cutting, resecting, or otherwise treating tissue. Aspects of the disclosure may provide the user with the ability to apply electrical energy or heat to tissue using a medical device having an electrode, and to deliver fluid into and/or under tissue with the same medical device. Aspects of the disclosure may provide the user with the ability to apply electrical energy or heat and also deliver fluid without having to switch or swap out end effectors. Aspects of the disclosure may help the user penetrate a layer of tissue (e.g., a submucosal layer) to cause perforation or otherwise cut, cauterize, or otherwise treat tissue. Aspects of the disclosure may help the user cut, resect, or otherwise remove tissue or other material without having to switch or swap out end effectors. Aspects of the disclosure may help the user to deflect a distal portion of the end effector.

Moreover, aspects of the disclosure may help the user to inflate or expand one or more balloons at the distal portion of the end effector, for example, helping to separate layers of tissue. For example, some aspects of the disclosure may help the user to inflate or expand one or balloons to expand or separate tissue layers and/or help to tension one or more tissue layers or tissue fibers. Tensioning one or more tissue layers or tissue fibers may help the user identify and/or distinguish between tissue layers, for example, between the mucosa (or mucosal) and muscularis layers and/or may help to reduce the possibility of inadvertent perforation of tissue. Some aspects of the disclosure may be used in performing an endoscopic, laparoscopic, arthroscopic, gynoscopic, thoracoscopic, cystoscopic, or other type of procedure.

Embodiments of the disclosure may relate to devices and methods for performing various medical procedures and/or treating portions of the large intestine (colon), small intestine, cecum, esophagus, stomach, any other portion of the gastrointestinal tract, lungs, and/or any other suitable patient anatomy. Various embodiments described herein include single-use or disposable medical devices. Some aspects of the disclosure may be used in performing an endoscopic, arthroscopic, bronchoscopic, ureteroscopic, colonoscopic, or other type of procedure. For example, the disclosed aspects may be used with duodenoscopes, bronchoscopes, ureteroscopes, colonoscopes, catheters, diagnostic or therapeutic tools or devices, or other types of medical devices. One or more of the elements discussed herein could be metallic, plastic, or include a shape memory metal (such as, e.g., nitinol), a shape memory polymer, a polymer, or any combination of biocompatible materials.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. Proximal and distal directions are labeled with arrows marked “P” and “D”, respectively, throughout various figures. As used herein, the terms “comprises,” “comprising,” “having,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

Reference will now be made in detail to examples of the disclosure described above and illustrated in the accompanying drawings. Wherever possible, the same reference numbers will be used throughout the drawings to refer to the same or like parts. It is noted that one or more aspects of the medical devices or methods discussed herein may be combined and/or used with one or more other aspects of the medical devices or methods discussed herein.

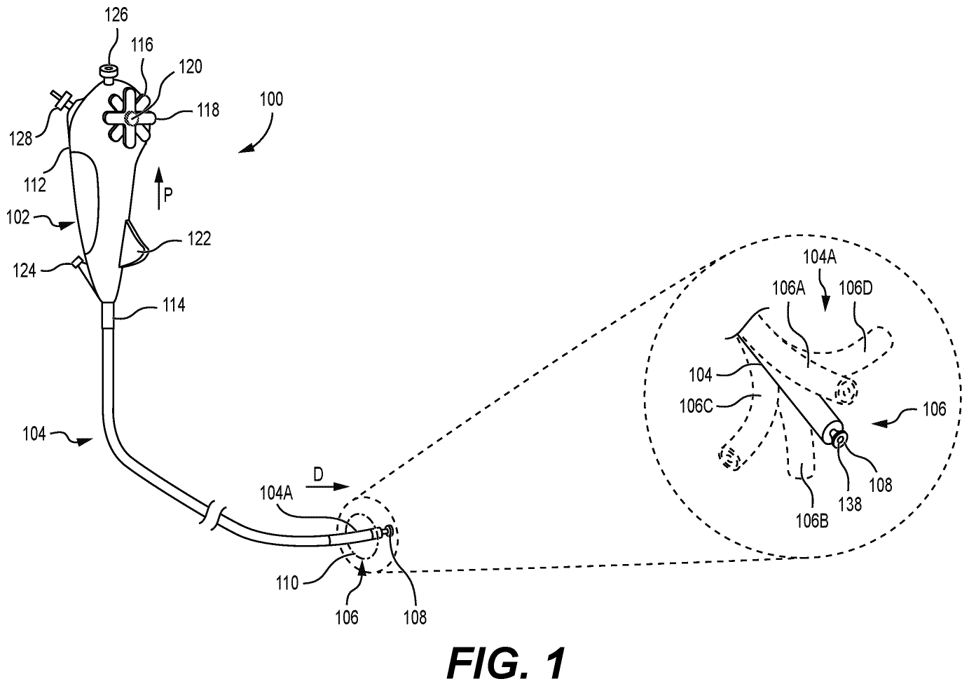

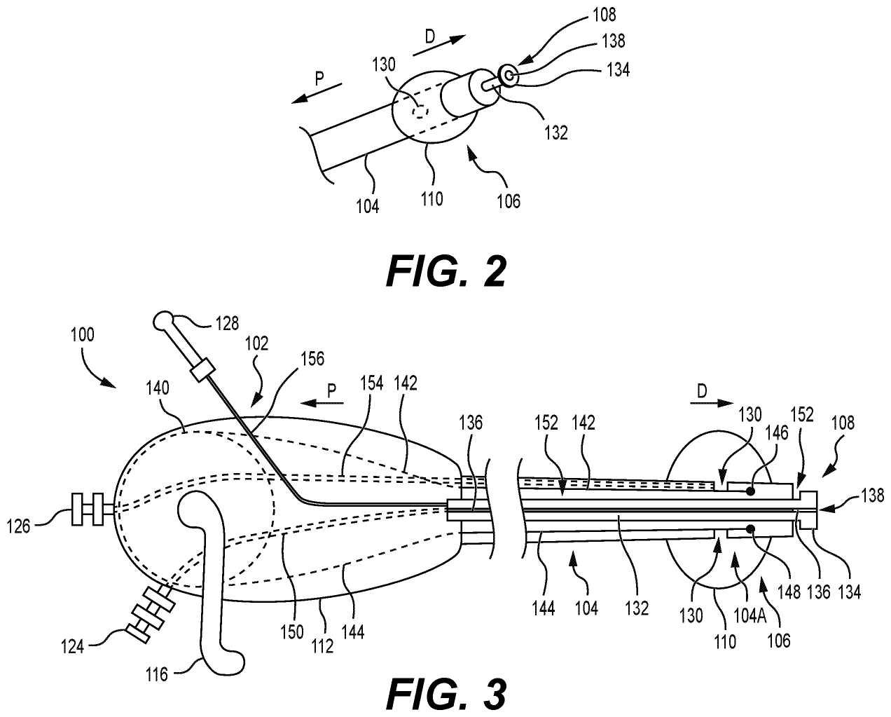

FIG. 1 illustrates a perspective view of an exemplary medical device 100 that includes a handle 102 and a shaft 104. Shaft 104 includes a distal end 106. Medical device 100 also includes an electrode 108, for example, at distal end 106. Electrode 108 may be movable relative to shaft 104, for example, extendable and/or retractable longitudinally relative to shaft 104. Electrode 108 may be energized to puncture, perforate, cut, resect, cauterize, remove, or otherwise treat tissue or other portions of a treatment site. Moreover, in some aspects, electrode 108 may include an electrode lumen 136 (FIG. 3) that terminates in an electrode outlet or electrode opening 138 to inject or otherwise deliver fluid to the treatment site, for example, into or between layers of tissue. In other aspects, however, electrode 108 may not include an electrode lumen, and thus may not provide for injection or other delivery of fluid. As discussed in detail below, handle 102 includes various connections and controls to control the position of distal end 106 and electrode 108. As shown in the enlarged portion of FIG. 1, at least a portion of distal end 106 of shaft 104 (e.g., deflectable portion 104A) is deflectable, and the deflection may be controlled by one or more portions of handle 102. Furthermore, as shown in FIGS. 1 and 2, distal end 106 includes one or more inflatable balloons 110, for example, extending radially outward from at least a portion of shaft 104 when inflated.

As shown in FIG. 1, handle 102 includes a handle body 112. A distal portion of handle body 112 may be coupled to a proximal portion of shaft 104 via a connector or strain relief element 114. Additionally, handle 102 includes one or more actuators or control mechanisms, for example, including one or more deflection elements or controls. The one or more deflection elements or controls may include one or more knobs or levers, for example, two knobs 116 and 118. Knobs 116 and 118 are movable (e.g., rotatable or pivotable) to control a deflection of at least a portion of shaft 104 (e.g., deflectable portion 104A. For example, knob 116 may be movable to control the deflection of at least a portion of shaft 104 (e.g., deflectable portion 104A) in a first plane (e.g., left/right), and knob 118 may be movable to control the deflection of at least a portion of shaft 104 (e.g., deflectable portion 104A) in a second plane (e.g., up/down). In some aspects, the first and second planes are substantially perpendicular, for example, to allow for four-way articulation of deflectable portion 104A of shaft 104, as shown in the enlarged portion of FIG. 1. As discussed in detail below, knobs 116 and 118 may be coupled to one or more respective wires (see, e.g., FIG. 3, as discussed below) to control the deflection of at least a portion of shaft 104. Knobs 116 and 118 may be coaxial, for example, each coupled to coaxial shafts or wheels. In some aspects, handle 102 includes a lock 120, for example, to lock or otherwise secure the position of one or more of knobs 116 and 118, which may help to lock or otherwise secure a position or orientation of at least a portion of shaft 104 (e.g., deflectable portion 104A). It is noted that one or more of knobs 116 and 118 may be one or more other actuators different from those shown in FIG. 1, for example, levers, sliders, etc.

It is noted that, in some aspects, handle 102 may only allow for two-way articulation (e.g., in one plane). For example, handle 102 may include only one knob or lever to control the two-way articulation. Alternatively, handle 102 may include two knobs or levers, with one knob or lever controlling movement in one way, and another knob or lever controlling movement in another (e.g., opposite) way.

Handle 102 may include a button, actuator, or trigger 122, for example, movable relative to a distal portion of handle body 112. Trigger 122 may be movable, for example, actuatable or depressible, to control a position (e.g., extension and/or retraction) and/or energization of electrode 108. For example, actuating or depressing trigger 122 may extend electrode 108 from distal end 106 of shaft 104. Trigger 122 may be biased (e.g., spring-biased), such that trigger 122 may return to the non-depressed position once pressure on trigger 122 is no longer applied. Returning to the non-depressed position may also retract electrode 108 to the non-extended position. Alternatively or additionally, in some aspects, depressing trigger 122 may control the energization of electrode 108. For example, depressing trigger 122 may energize electrode 108, and releasing pressure on trigger 122 may terminate the energizing of electrode 108.

Handle 102 also includes one or more connections and/or ports, for example, extending from or otherwise coupled to handle body 112. For example, handle 102 may include one or more of an electrode fluid port 124, a balloon fluid port 126, and/or an electrical connection 128. As discussed below, electrode fluid port 124 and balloon fluid port 126 may be fluidly connected to electrode 108 and balloon 110, respectively, for example, via respective lumens in handle 102 and shaft 104. Electrical connection 128 may be electrically connected to electrode 108, for example, via one or more conductive elements (e.g., wires, cables, filaments, rods, etc.) in handle 102 and shaft 104.

Electrode fluid port 124 may be positioned on a distal portion of handle body 112, for example, adjacent and/or opposing trigger 122. Balloon fluid port 126 and/or electrical connection 128 may be positioned on respective proximal portions of handle body 112, for example, adjacent or proximal to knobs 116 and 118. Electrode fluid port 124 may receive fluid (e.g., water, Orise™ gel, saline, etc.) to be delivered via a lumen in electrode 108, for example, between two layers of tissue or otherwise to the treatment site. Balloon fluid port 126 may receive fluid (e.g., air, water, etc.) to be delivered to balloon 110, for example, to inflate balloon. The delivery of the fluid to each of electrode fluid port 124 and balloon fluid port 126 may be controlled by a fluid delivery device, for example, a syringe, pump, etc. Moreover, electrical connection 128 may receive energy (e.g., cautery energy) from an energy source to be delivered to electrode 108, for example, to cut, cauterize, penetrate, perforate, or otherwise treat tissue. In some aspects, the delivery of energy to electrical connection 128 may be controllable, for example, via a separate button or trigger, one or more foot pedals, etc.

Furthermore, although not shown, handle 102 may include a strap or other connection element, for example, coupled to or otherwise extending from handle body 112. In some aspects, the strap or other connection element may help to allow for handle body 112 to be coupled to another medical device, for example, to a handle or other portion of a scope or other insertion device.

As mentioned, shaft 104 includes steerable or deflectable portion 104A, for example, at distal end 106 of shaft 104. Deflectable portion 104A may be, for example, an articulation joint. Shaft 104 and deflectable portion 104A may include a variety of structures which are known or may become known in the art. As mentioned, movement of knobs 116 and 118 control the deflection of deflectable portion 104A, and thus the deflection of distal end 106. For example, as shown in the enlarged portion of distal end 106 of shaft 104, movement of knob 116 may deflect deflectable portion 104A in a first plane (e.g., up and down), for example, between at least positions or orientations of distal ends 106A and 106B. Moreover, movement of knob 118 may deflect deflectable portion 104A in a second plane (e.g., left and right), for example, between at least positions or orientations of distal ends 106C and 106D. In some aspects, the first plane and the second plane are substantially perpendicular. Furthermore, in some aspects, movement of one or more of knobs 116 and 118 may allow for deflectable portion 104A, including distal end 106, to be deflected through a substantially semi-spherical range of motion. In other aspects, movement of one or more of knobs 116 and 118 may allow for distal end 106 to be deflected through a smaller range of motion (e.g., through a substantially conical range of motion) or through a larger range of motion (e.g., through an approximately spherical range of motion). It is noted that, for clarity, electrode 108 is omitted on distal end 106 in the illustration of distal end 106 in the respective deflected positions or orientations in the enlarged portion of FIG. 1 (e.g., positions or orientations of distal ends 106A, 106B, 106C, and 106D).

FIG. 2 illustrates distal end 106 of shaft 104 with balloon 110 inflated. For example, a distal portion of balloon 110 may be sealingly coupled to a portion of shaft 104, and a proximal portion of balloon 110 may be sealingly coupled to another portion of shaft 104. As shown, balloon 110 may radially surround a portion of shaft 104. For example, balloon 110 may extend radially around an entire circumference of the portion of shaft 104. Alternatively, although not shown, balloon 110 may extend radially around only a portion of the circumference of shaft 104. In any of these aspects, shaft 104 includes at least one balloon hole 130, for example, in an outer surface of a distal portion of shaft 104. Balloon hole 130 fluidly couples balloon fluid port 126 and any balloon lumens in shaft 104 to balloon 110. In these aspects, fluid (e.g., air, water, etc.) may be delivered through balloon fluid port 126, through a lumen in handle 102, through a lumen in shaft 104, and out of balloon hole 130 to inflate balloon 110. It is noted that, as shown in FIGS. 1-3, balloon 110 is positioned on shaft 104 at a position proximal of electrode 108 and the distalmost end of distal end 106 of shaft 104. Stated another way, balloon 110 is proximal of electrode 108 (and electrode 108 is distal to balloon 110), both when electrode 108 is extended distally and when electrode 108 is retracted proximally. Furthermore, in some aspects, balloon 110 may be proximal of deflectable portion 104A of shaft 104. For example, a distal end of balloon 110 may be adjacent to a proximal end of deflectable portion 104A of shaft 104. In these aspects, balloon 110 (and the portion of shaft 104 that balloon 110 surrounds) may remain relatively stationary and/or fixed in position within the body lumen of the subject (e.g., helping to apply tension to and/or separate tissue(s)), while deflectable portion 104A of shaft 104 and/or electrode 108 move within the body lumen (e.g., while deflectable portion 104A deflects or sweeps and/or while the electrode 108 is extended and/or retracted).

In some aspects, balloon 110 may inflatable to a size that is substantially the same size or smaller than a diameter of a respective bodily lumen (e.g., the esophagus, if distal end 106 of shaft 104 is configured to be delivered into the esophagus). For example, in a deflated state, balloon 110 may abut shaft 104 or otherwise approximate the size of shaft 104. Moreover, in an inflated state, balloon 110 may include a diameter of approximately 3 mm to approximately 30 mm, for example, approximately 15 mm. In the inflated state, balloon 110 may include a length of approximately 5 mm to approximately 30 mm, for example, approximately 15 mm. At least a portion of balloon 110 (i.e., in the inflated state) may include an approximately circular shape (e.g., in a lateral cross-section). Alternatively or additionally, at least a portion of balloon 110 (i.e., in the inflated state) may include an approximately ovular or elliptical shape (e.g., in the lateral cross-section) with a major axis and a minor axis. In some aspects, the major axis of an elliptical balloon could range from approximately 5 mm to approximately 30 mm (e.g., approximately 20 mm), and the minor axis could range from approximately 5 mm to approximately 30 mm (e.g., approximately 15 mm). Furthermore, in some aspects, balloon 110 may include an at least partially conical shape, for example, with one or more tapers or funnels from a smaller diameter to a larger diameter (e.g., with a distal portion of balloon 110 being smaller than a proximal portion of balloon 110, or vice versa). Additionally, in some aspects, suction or negative pressure may be applied to balloon fluid port 126, for example, to deflate balloon 110. Balloon 110 may be formed of a flexible, medically-safe material (e.g., plastic or silicone material or a combination thereof, for example, nylon, Pebax, a nylon/Pebax blend, latex, polyethylene terephthalate (PET), etc.).

Although not shown in FIG. 2, shaft 104 may include a plurality of balloon holes 130, for example, fluidly connected to a plurality of balloons 110. The plurality of balloons 110, and corresponding balloon holes 130, may be positioned longitudinally along shaft 104. Alternatively, the plurality of balloons 110, and corresponding balloon holes 130, may be positioned radially around the circumference of shaft 104. In these aspects, the plurality of balloon holes 130 may be fluidly connected to the same balloon lumen in shaft 104, for example, such that the plurality of balloons 110 are inflated and/or deflated at the same time and/or rate. In another aspects, the plurality of balloon holes 130 (or a subset of the plurality of balloon holes 130) may be fluidly connected to different balloon fluid ports 126 and/or to different balloon lumens in handle 102 and/or shaft 104, for example, such that the plurality of balloons 110 (or a subset of the plurality of balloons) are inflated or deflated at different times and/or different rates.

Furthermore, although not shown, shaft 104 may include a plurality of balloon lumens coupled to respective balloon holes 130. Respective balloon lumens may be coupled to one or more fluid or inflation sources (e.g., air, water, etc.) and/or deflation sources (e.g., suction), for example, via respective balloon fluid ports 126. In these aspects, balloon(s) 110 may be inflated via one or more balloon fluid ports 126, one or more balloon lumens, and one or more balloon holes 130, and balloon(s) 110 may be deflated via one or more balloon fluid ports 126, one or more other balloon lumens, and one or more other balloon holes 130.

As shown in FIGS. 2 and 3, electrode 108 includes an electrode shaft 132 and a distal end 134. Electrode shaft 132 may be generally cylindrical, for example, with a constant width. Alternatively, although not shown, electrode shaft 132 may include one or more tapered portions, for example, between portions that include different widths. Distal end 134 may be wider (e.g., extending radially outward relative to a longitudinal axis of electrode 108) than electrode shaft 132. In this aspect, distal end 134 may remain extended and exposed distal to distal end 106 in a retracted position. Distal end 134 may include a flat distalmost face, as shown. Alternatively, distal end 134 may include a rounded (e.g., a half-ball or semi-spherical) distal end. Although not shown, one or more portions of electrode 108 (e.g., distal end 134) may include one or more insulating materials. Alternatively, an entirety of electrode 108 may be conductive.

Moreover, as mentioned above, in some aspects, electrode 108 includes electrode lumen 136 that terminates distally in electrode opening 138. In these aspects, fluid (e.g., water, Orise™ gel, saline, etc.) may be delivered through electrode fluid port 124 (e.g., from a syringe or other fluid source), a lumen or tube in handle 102, a lumen or tube in shaft 104, and electrode lumen 136 to be delivered out of electrode opening 138. The fluid may be delivered to tissue (e.g., injected between layers of tissue) or otherwise delivered to the treatment site. Alternatively, in other aspects, electrode 108 does not include electrode lumen 136 or electrode opening 138.

FIG. 3 is a schematic view of various portions of medical device 100. As discussed above, medical device 100 includes handle 102, with handle body 112, and shaft 104 extending from handle body 112. It is noted that strain relief element 114 and trigger 122 are omitted for clarity. Furthermore, only one knob 116 is shown for clarity, with knob 116 being represented by a lever in order to illustrate the internal portions of handle 102.

As mentioned above, handle 102 includes at least one knob 116 and a plurality of fluid or electrical connections. As shown, knob 116 is coupled to a rotatable or pivotable element, for example, a wheel 140, or a pulley, such that rotation of knob 116 rotates wheel 140. Additionally, one or more pull wires, for example, two pull wires 142 and 144, may be coupled to and extend distally from wheel 140. Each of pull wires 142, 144 may extend through handle body 112 and through respective portions of shaft 104, for example, terminating distally at distal couplings 146, 148 in interior portions of distal end 106 of shaft. Each of pull wires 142, 144 may be secured at distal couplings 146, 148. Distal couplings 146 may be offset from one another (e.g., 180 degrees apart, 90 degrees apart, or any other suitable amount apart). In these aspects, rotation of knob 116 rotates wheel 140, such that the rotation of wheel 140 pulls or urges one of pull wires 142 or 144 proximally, for example, to help deflect deflectable portion 104A of shaft 104. For example, rotating wheel 140 in a first direction may pull or urge pull wire 142 proximally, to help deflect deflectable portion 104A of shaft 104 in a first deflection direction, and rotating wheel 140 in a second direction (e.g., opposite to the first direction) may pull or urge pull wire 144 proximally, to help deflect deflectable portion 104A of shaft 104 in a second deflection direction (e.g., opposite to the first deflection direction). Although not shown, other and/or additional deflection or articulation mechanisms may also or instead be incorporated in medical device 100 to control the position of distal end 106 of shaft 104, for example, by controlling a position and/or orientation of deflectable portion 104A of shaft 104.

Additionally, handle 102 may include electrode fluid port 124, balloon fluid port 126, and electrical connection 128. As shown, electrode fluid port 124 is in fluid communication with electrode 108 via one or more electrode fluid lumens or tubes 150, for example, extending at least from electrode fluid port 124 to electrode 108 to fluidly connect electrode fluid port 124 and electrode lumen 136. It is noted that FIG. 3 illustrates electrode 108 extending proximally to handle body 112. This disclosure is not so limited. For example, electrode 108 may be positioned within a shaft lumen 152 of shaft 104 at a position spaced distally from handle body 112. In this aspect, electrode fluid tube(s) 150 may extend through a portion of shaft 104 in order to fluidly connect electrode fluid port 124 with electrode lumen 136. Furthermore, in some aspects, handle 102 may include a first electrode fluid lumen, and shaft 104 may include a second electrode fluid lumen, for example, fluidly connected to the first electrode fluid lumen, in order to fluidly connect electrode fluid port 124 to electrode lumen 136. Additionally, in some aspects, one or more of electrode fluid port 124 and electrode fluid tube(s) 150 may be movable, for example, to impart extension and/or retraction of electrode 108 relative to distal end 106 of shaft 104. Alternatively, other actuation mechanisms (e.g., wires, rods, or the like) may be utilized to extend/retract electrode 108.

Moreover, balloon fluid port 126 is in fluid communication with balloon 110 via one or more balloon fluid lumens or tubes 154. As shown in FIG. 3, one or more balloon fluid tubes 154 may extend from balloon fluid port 126, through handle body 112, and through at least a portion of shaft 104 to balloon hole(s) 130. Balloon fluid tube 154 may extend through a separate lumen in shaft 104. For example, electrode 108 may be positioned within a first lumen or working channel in shaft 104, and balloon fluid tube 154 may be positioned within a second lumen or auxiliary channel in shaft 104. Additionally, balloon fluid tube 154 may extend to more than one balloon hole 130, for example, two balloon holes 130. As shown in FIG. 3, the two balloon holes 130 may be positioned on circumferentially opposite sides of distal end 106 of shaft 104. For example, a distal portion of balloon fluid tube 154 may include a branching or bifurcation, such that fluid delivered or suction applied through balloon fluid tube 154 is delivered/applied to more than one balloon holes 130 in order to inflate or deflate balloon(s) 110. Moreover, in some aspects, handle 102 may include a first balloon fluid tube, and shaft 104 may include a second balloon fluid tube, for example, fluidly connected to the first balloon fluid tube, in order to fluidly connect balloon fluid port 126 to balloon hole(s) 130 and balloon(s) 110. In some aspects, handle 102 may include balloon fluid tube 154, and balloon fluid tube 154 may be fluidly connected to a lumen (e.g., an auxiliary lumen or channel) in shaft 104. The lumen in shaft 104 is then fluidly connected to balloon hole(s) 130.

Furthermore, electrical connection 128 is electrically connected to electrode 108 via one or more conductive wires or elements 156. As shown in FIG. 3, one or more conductive elements 156 (e.g., wires, cables, filaments, rods, etc.) may extend from electrical connection 128, through a portion of handle body 112, and to electrode 108. As discussed above, it is noted that FIG. 3 illustrates electrode 108 extending proximally to handle body 112. This disclosure is not so limited. For example, electrode 108 may be positioned within shaft lumen 152 of shaft 104 at a position spaced distally from handle body 112. In this aspect, conductive element 156 may extend through a portion of shaft 104 in order to electrically connect electrical connection 128 with electrode 108. Furthermore, in some aspects, handle 102 may include a first conductive element, and shaft 104 may include a second conductive element, for example, electrically connected to the first conductive element, in order to electrically connect electrical connection 128 to electrode 108. Moreover, in some aspects, conductive element 156 may be movable relative to one or more of handle 102 and/or shaft 104, for example, via action on trigger 122. In these aspects, movement of conductive element 156 may impart extension and/or retraction of electrode 108 relative to distal end 106 of shaft 104.

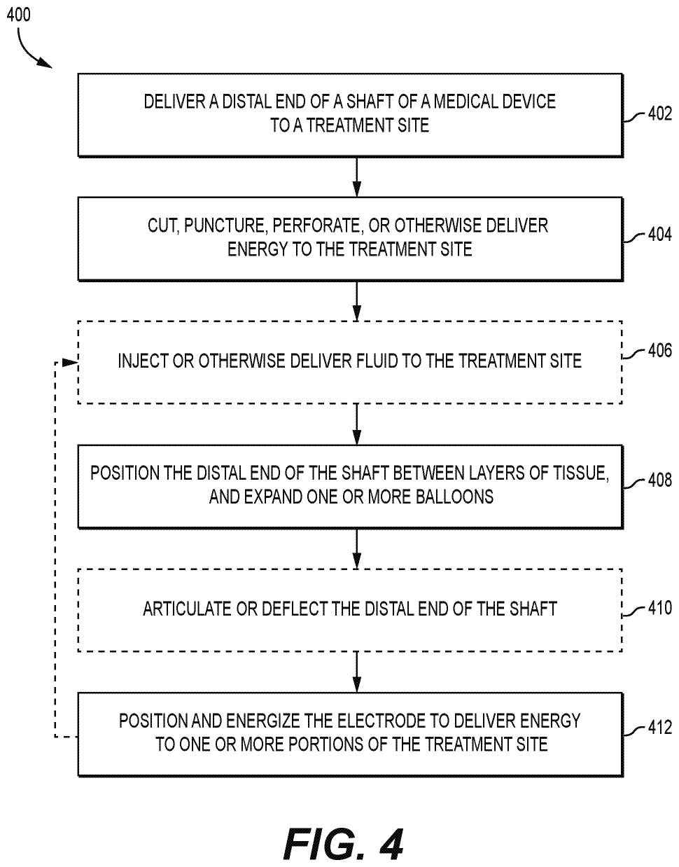

FIG. 4 illustrates a method 400 that may be performed with medical device 100 or portions of medical device 100 discussed herein. Specifically, method 400 includes an initial step 402 that includes delivering distal end 106 of shaft 104 to a treatment site. Shaft 104 may be delivered to the treatment site via an insertion device (e.g., an endoscope, insertion device, sheath, etc.) through a surgical incision or a natural anatomical orifice. In some aspects, shaft 104 may be delivered through the subject's mouth and into the subject's esophagus, stomach, and/or other portion of the GI tract. In some aspects, a distal portion of the insertion device may be deflectable, for example, to help position distal end 106 of medical device 100 relative to the treatment site. Additionally, step 402 may include positioning distal end 106 at the treatment site. Step 402 may include extending electrode 108 distally, for example, via trigger 122. Moreover, step 402 may include deflecting deflectable portion 104A of shaft 104, for example, via one or more of knobs 116, 118.

Next, method 400 includes a step 404 that includes cutting, puncturing, perforating, or otherwise delivering energy to the treatment site, for example, with electrode 108. As mentioned above, electrode 108 may be energized by depressing trigger 122, which may also extend electrode 108. Alternatively or additionally, electrode 108 may be energized by actuating one or more other buttons, triggers, foot pedals, etc. Delivering the energy to the treatment site may help to cut, puncture, perforate, or otherwise treat one or more layers of tissue at the treatment site, for example, the mucosa in a portion of the subject's GI tract.

An optional step 406 may include injecting or otherwise delivering fluid to the treatment site. For example, optional step 406 may include positioning a portion of electrode 108 (e.g., distal end 134) adjacent to or at least partially into the cut, punctured, or perforated tissue, and delivering fluid through electrode lumen 136 and out of electrode opening 138. As mentioned, in some aspects, electrode 108 may be fluidly connected to electrode fluid port 124. In some aspects, injecting or otherwise delivering fluid to the treatment site may include coupling a syringe or other fluid delivery device to electrode fluid port 124 and delivering fluid (e.g., water, Orise™ gel saline, etc.) through electrode fluid port 124 and electrode fluid tube 150. The fluid may be delivered between layers of tissue (e.g., into the submucosal space between the mucosa and the muscularis layers) to help separate the layers of tissue.

Next, step 408 includes positioning the distal end of shaft 104 between layers of tissue, and expanding one or more balloons 110. In this aspect, with distal end 106 positioned between layers of tissue (e.g., between the mucosa and the muscularis), expanding one or more balloons 110 may help to more quickly and/or more effectively separate and/or tension the layers of tissue and/or tissue fibers, for example, via blunt dissection. Expanding one or more balloons 110 may also help to stabilize and/or provide traction to distal end 106 of shaft 104, for example, when positioning distal end 106. Expanding one or more balloons 110 may further help to stabilize and/or provide traction to distal end 106 of shaft 104, for example, when delivering energy and/or fluid via electrode 108. Furthermore, in some aspects, one or more balloons 106 may help the user identify and/or distinguish between tissue layers, for example, between the mucosa (or mucosal) and muscularis layers and/or may help to reduce the possibility of inadvertent perforation of tissue.

An optional step 410 includes articulating or deflecting the distal end of the shaft. In this aspect, articulating or deflecting distal end 106 (e.g., via control of deflectable portion 104A of shaft 104 with one or more of knobs 116, 118) with distal end 106 between the layers of tissue may further help to more quickly and/or effectively separate the layers of tissue. Additionally, the articulation or deflection may be performed with the one or more balloons 110 inflated, further aiding in the separation of the layers of tissue. As mentioned, the one or more balloons 110 are proximal of electrode 108 and the distal end of shaft 104, both with electrode 108 extended and with electrode 108 retracted. Additionally, the one or more balloons 110 may at least partially overlap (i.e., longitudinally) with deflectable portion 104A of shaft 104, such that the one or more balloons 110 at least partially move with deflectable portion 104A as deflectable portion 104A is deflected (e.g., via one or more of knobs 116, 118). Alternatively, the one or more balloons 110 may be proximal of deflectable portion 104A of shaft 104 (e.g., with a distal end of balloon(s) 110 being proximal of or adjacent to a proximal end of deflectable portion 104A), such that the one or more balloons 110 remain stationary or fixed within the body lumen as deflectable portion 104A is deflected.

Moreover, in some aspects, step 410 may include advancing or urging distal end 106 distally (e.g., in addition to articulation or deflection) to further separate the layers of tissue, for example, via blunt tissue dissection. The position of distal end 106 may help to maintain the separation of the layers of tissue. Furthermore, inflation of the one or more balloons 110 may help to maintain the separation and/or tensioning of the layers of tissue. Additionally, in some aspects, additional fluid (e.g., water, Orise™ gel, saline, etc.) may be delivered through electrode 108, which may also help to maintain the separation of the layers of tissue.

Furthermore, a step 412 includes positioning and energizing the electrode to deliver energy to one or more portions of the treatment site. In some aspects, the one or more portions of the treatment site may be one or more portions of one or more of the separated layers of tissue. Electrode 108 may be extended and/or retracted relative to distal end 106 of shaft 104, for example, via trigger 122. Additionally, electrode 108 may be positioned via the deflection of distal end 106 of shaft 104 (e.g., via control of deflectable portion 104A with one or more of knobs 116, 118). Moreover, if shaft 104 is delivered through a scope of insertion device, the distal portion of the scope or insertion device may be deflectable to help position distal end 106 of shaft 104 and/or electrode 108.

Moreover, as shown in FIG. 4, in some aspects, method 400 may optionally include repeating one or more steps 402-412. For example, as shown in FIG. 4, after step 412, method 400 may include repeating one or more of steps 406-412. In these aspects, after step 412, the user may again perform one or more of: optional step 406 to inject or otherwise deliver fluid to the treatment site, step 408 to position the distal end of the shaft between layers of tissue and expand one or more balloons, optional step 410 to articulate or deflect the distal end of the shaft, and/or step 412 to position and energize the electrode to deliver energy to one or more portions of the treatment site. Repeating one or more of the steps of method 400 may help to tunnel through or otherwise separate additional tissue or layers of tissue.

The separation and/or tensioning of the layers of tissue, as discussed above, may help to allow for the positioning of electrode 108, including the positioning of distal end 106. The separation and/or tensioning of the layers of tissue may also help to allow electrode 108 to be positioned in a field of view, for example, of the scope or insertion device without having to reposition or move the scope or insertion device. As discussed, electrode 108 may be energized via electrical connection 128 and conductive element 156. The energized electrode 108 may be applied to one or more portions of the treatment site to cut, dissect, ablate, mark, coagulate, cauterize, or otherwise treat the treatment site. Furthermore, in some aspects, step 412 may include delivering fluid into or between additional layers of tissue, for example, to help raise or separate layers of tissue for removal or treatment. In these aspects, electrode 108 may be used to deliver energy and deliver fluid to the treatment site without having to remove or swap out electrode 108 for another end effector.

Various aspects discussed herein may allow for a medical device (e.g., medical device 100) to be delivered to a treatment site, for example, alone or via a scope or insertion device, to perform endoscopic submucosal dissection (“ESD”) or otherwise treat a treatment site. Various aspects discussed herein may allow for a user to perform a “third space” procedure, for example, a “third space” endoscopy procedure. Additionally, various aspects discussed herein may help to improve the efficacy of treatment and/or recovery from a procedure, for example, a procedure to treat a treatment side. Various aspects discussed herein may help to reduce and/or minimize the duration of the procedure, and/or may help reduce risks of inadvertent contact with tissue or other material during delivery, repositioning, or removal of a medical device for the procedure.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical device, comprising:
a handle, including:
an electrical connection; and
a balloon fluid port;
a shaft extending from a distal portion of the handle;
an electrode positioned at a distal end of the shaft, wherein the electrode is electrically connected to the electrical connection via one or more conductive elements extending through the handle and the shaft; and
at least one inflatable balloon positioned on a portion of shaft, wherein the at least one inflatable balloon is fluidly coupled to the balloon fluid port via one or more tubes extending through the handle and the shaft.

2. The medical device of claim 1, wherein the handle further includes one or more deflection elements, and wherein at least a portion of the shaft is deflectable via movement of the one or more deflection elements.

3. The medical device of claim 2, wherein the one or more deflection elements are each coupled to a wheel that is connected to one or more pull wires that extend through the handle and are secured to one or more interior portions of the shaft such that movement of the one or more deflection elements urges the one or more pull wires proximally to deflect a portion of the shaft.

4. The medical device of claim 3, wherein the one or more deflection elements includes two coaxial deflection knobs, wherein each of the two deflection knobs controls a deflection of the portion of the shaft in substantially perpendicular planes.

5. The medical device of claim 4, wherein the portion of the shaft is deflectable in a substantially semi-spherical range of motion, and
wherein the portion of the shaft is deflectable with the at least one inflatable balloon inflated.

6. The medical device of claim 4, wherein the handle further includes one or more locks to secure a position of one or more of the two deflection knobs.

7. The medical device of claim 1, wherein the electrode includes an electrode lumen, wherein the handle further includes an electrode fluid port, and wherein the electrode fluid port is fluidly coupled to the electrode lumen via one or more electrode fluid tubes that are configured to deliver a fluid to the electrode lumen from the electrode fluid port.

8. The medical device of claim 7, wherein the electrode includes an electrode shaft and a distal end, wherein the distal end is wider relative to a longitudinal axis than the electrode shaft.

9. The medical device of claim 7, wherein the handle further includes a trigger, and wherein the electrode is extendable distally and retractable proximally relative to the distal end of the shaft via actuation of the trigger.

10. The medical device of claim 9, wherein the distal end of the electrode remains positioned distally of the distal end of the shaft when the electrode is in a retracted position.

11. The medical device of claim 9, wherein the trigger is positioned on a distal portion of the handle, wherein the electrode fluid port is positioned on a distal portion of the handle, and wherein the electrical connection and the balloon fluid port are positioned on a proximal portion of the handle.

12. The medical device of claim 7, wherein an entirety of the electrode is conductive.

13. The medical device of claim 1, wherein the at least one inflatable balloon is fluidly coupled to one or more tubes via at least one balloon hole in an outer surface of a distal portion of the shaft.

14. The medical device of claim 13, wherein the at least one balloon hole includes two balloon holes positioned on circumferentially opposite sides of the shaft.

15. The medical device of claim 2, wherein the medical device is configured to perform a third space endoscopy procedure.

16. A medical device, comprising:
a handle, including:
an electrical connection; and
one or more deflection controls;
a shaft extending from a distal portion of the handle; and
an electrode positioned at a distal end of the shaft, wherein the electrode is electrically connected to the electrical connection via one or more conductive elements extending through the handle and the shaft;
wherein at least a portion of the shaft is deflectable via movement of the one or more deflection controls.

17. The medical device of claim 16, wherein the electrode includes an electrode shaft and a distal end, wherein the distal end is wider relative to a longitudinal axis than the electrode shaft, and wherein the electrode includes an electrode lumen,
wherein the handle further includes an electrode fluid port, and wherein the electrode fluid port is fluidly coupled to the electrode lumen via one or more electrode fluid tubes that are configured to deliver a fluid to the electrode lumen from the electrode fluid port.

18. The medical device of claim 17, further comprising:
at least one inflatable balloon positioned on a distal portion of the shaft,
wherein the handle further includes a balloon fluid port, wherein the at least one inflatable balloon is fluidly coupled to the balloon fluid port via one or more tubes extending through the handle and the shaft.

19. A method of treating a treatment site, comprising:
delivering a distal end of a shaft of a medical device to a treatment site, wherein the shaft includes a deflectable portion, wherein an electrode is movably positioned at the distal end of the shaft;
delivering energy to the treatment site to cut, puncture, or perforate one or more layers of tissue at the treatment site;
positioning the distal end of the shaft between layers of tissue, and expanding one or more balloons to separate layers of tissue, wherein the one or more balloons are positioned on a distal portion of the shaft and are fluidly connected to one or more balloon fluid ports via one or more balloon fluid tubes and one or more balloon holes; and
positioning and energizing the electrode to deliver energy to one or more portions of the treatment site, wherein the one or more portions of the treatment site are between separated layers of tissue.

20. The method of claim 19, wherein the electrode includes an electrode lumen fluidly connected to a fluid source, and wherein the method further comprises:
before positioning the distal end of the shaft between layers of tissue, injecting or otherwise delivering fluid to the treatment site through the electrode lumen to separate layers of tissue; and
before positioning and energizing the electrode to delivery energy to one or more portions of the treatment site, articulating or deflecting the distal end of the shaft to further separate the layers of tissue.
